(19) United States
(12) Patent Application Publication
(10) Pub. No.: US 2003/0049287 A1
(43) Pub. Date: Mar. 13, 2003

(54) Catechol oximes and their use in cosmetic and dermatological preparations

(76) Inventors: Jakob Peter Ley, Holzminden (DE); William Johncock, Hoxter (DE); Johannes Kaulen, Odenthal (DE)

Correspondence Address: BAYER CORPORATION, PATENT DEPARTMENT, 100 BAYER ROAD, PITTSBURGH, PA, 15205, US

(21) Appl. No.: 10/149,495

(22) PCT Filed: Dec. 1, 2000
(86) PCT No.: PCT/EP00/12111

(30) Foreign Application Priority Data
Dec. 14, 1999 (DE) 199 60 105.4

(51) Int. Cl. A61K 31/54; A61K 31/537; A61K 31/495; A61K 31/445; A61K 7/00; A61K 31/40; A61K 31/15

(52) U.S. Cl. 424/401; 514/227.5; 514/238.5; 514/252.12; 514/317; 514/408; 514/640

(57) ABSTRACT

The invention relates to cosmetic and/or dermatological preparations which contain, in part, novel catechol oximes of formula (I). Said preparations can promote, in physiological systems, the natural defense mechanisms against free radicals and reactive oxygen compounds, or can be used as protective agents in cosmetic or pharmaceutical products whose oxidation-sensitive constituents should be protected from autooxidation.

The invention relates to cosmetic and dermatological preparations which comprise catechol oximes, some of which are new. The invention also relates to the use of these catechol oximes, some of which are new, in cosmetic and/or dermatological preparations. The invention additionally relates to the use of these preparations for protecting cells and tissue in mammals from the harmful effects of free radicals and reactive oxygen compounds which promote ageing.

For cosmetic and/or dermatological preparations, active ingredients are sought which, in physiological systems, in particular in or on the skin, the nails or hair of mammals, aid the natural defense mechanisms against free radicals and reactive oxygen compounds or, as protective substances in cosmetics, pharmaceuticals or foods, protect their oxidation-sensitive constituents against autoxidation.

Antioxidants are substances which, in small concentrations compared with the oxidizable substrate, significantly delay oxidation or prevent it completely. Many antioxidants function at the same time as free-radical scavengers and/or as complexing agents for heavy metal ions.

Formulations for protecting against photo damage, comprising 0.1 to 2% of 2-hydroxyphenyloximes as chelating active ingredients, have been proposed in WO 95 01,157. In the application it is emphasized that for the claimed effect the hydroxyl group must be in the ortho position relative to the oxime group. However, the examples given in the application do not have an antioxidative effect or have only a negligible antioxidative effect, particularly in oxidative systems in which metal ions do not play a prooxidative role.

We have found cosmetic and/or dermatological preparations which comprise catechol oximes of the formula

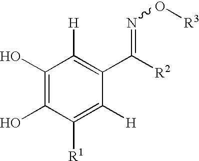

where

R¹ is hydrogen, lower alkyl or the group —O—R⁴ in which R⁴ is hydrogen or lower alkyl, and

R² is hydrogen, an alkyl radical having 1 to 22 carbon atoms or an alkenyl radical having 2 to 22 carbon atoms, and

R³ is hydrogen, an optionally substituted alkyl radical having 1 to 22 carbon atoms, an optionally substituted alkenyl radical having 2 to 22 carbon atoms, an optionally substituted aryl or arylalkyl radical having 6 to 12 carbon atoms or an optionally substituted heterocyclyl or heterocyclylalkyl radical having 2 to 12 carbon atoms and at least one atom from the group oxygen, sulfur or nitrogen,

including stereoisomers thereof or mixtures thereof.

The cosmetic and/or dermatological preparations according to the invention aid, in physiological systems, e.g. the skin, the hair or the nails, the natural defense mechanisms against free radicals and reactive oxygen compounds and protect in cosmetics, pharmaceuticals or foods, their oxidation-sensitive constituents against autoxidation or photooxidation.

Surprisingly, it has been found that the catechol oximes according to the invention are very good free-radical scavengers and particularly strong antioxidants. They are preferably suitable as antioxidants for lipids. In particular, the catechol oximes according to the invention are able to suppress the harmful effects of free radicals and/or oxidative processes which are induced by UV light, on and/or in the human skin, and to aid natural antioxidative processes.

Lower alkyl in the catechol oximes according to the invention is generally a short-chain saturated, straight-chain, cyclic or branched hydrocarbon radical having, preferably, 1 to 4 carbon atoms. Specifically, mention may be made of: methyl, ethyl, n-propyl, isopropyl, cyclopropyl, n-butyl, sec-butyl, isobutyl, tert-butyl, cyclopropylmethyl or the various isomers of the methylcyclopropyl radical. Particular preference is given to methyl and ethyl.

Alkyl having 1 to 22 carbon atoms is generally a saturated, straight-chain, cyclic or branched hydrocarbon radical. The radical preferably contains 1 to 10 carbon atoms. Specifically, mention may be made of: methyl, ethyl, n-propyl, isopropyl, n-butyl, sec-butyl, isobutyl, tert-butyl, the respective various straight-chain or branched isomers of the pentyl, hexyl, heptyl, octyl, nonyl and decyl radical, cyclopentyl, cyclopentylmethyl, cyclopentylethyl, cyclopentylpropyl, the various isomers of the methylcyclopentyl radical, cyclohexyl, cycloheptyl, cyclooctyl, menthyl, isomenthyl, homomenthyl, norbornyl, bornyl. Particular preference is given to methyl, ethyl, n-propyl, isopropyl, cyclopropyl, n-butyl, sec-butyl, isobutyl, tert-butyl, cyclopentyl, cyclohexyl, menthyl, n-pentyl, n-hexyl, n-heptyl, n-octyl, n-nonyl and n-decyl.

Alkenyl having 2 to 22 carbon atoms is generally an unsaturated straight-chain, cyclic or branched hydrocarbon radical. The radical preferably contains 2 to 10 carbon atoms. Specifically, mention may be made of: ethenyl, 1- or 2-propenyl, 1-, 2- or 3-butenyl, 2-methyl-1-propenyl, 2-methyl-2-propenyl, 1,3-butadienyl, 1,3-pentadienyl, 1,4-pentenyl, 2,4-pentenyl, the respective various straight-chain, cyclic or branched isomers of the pentenyl, hexenyl, heptenyl, octenyl, nonenyl and decenyl radicals. Particular preference is given to ethenyl, 1- or 2-propenyl, 1-, 2- or 3-butenyl, 2-methyl-1-propenyl, 2-methyl-2-propenyl, 3-methyl-1-pentenyl, 3-methyl-2-pentenyl, 3-methyl-3-pentenyl, cyclopentenyl, cyclohexenyl, pinenyl, norbornenyl and bornenyl.

Aryl having 6 to 12 carbon atoms is generally an aromatic hydrocarbon radical. Preference is given to phenyl and naphthyl. Particular preference is given to phenyl.

Arylalkyl having 6 to 12 carbon atoms is generally an alkyl radical substituted by aryl. Preference is given to arylalkyl radicals having in total 7 to 12 carbon atoms. Particular preference is given to phenylmethyl, 1- or 2-phenylethyl, 1-, 2- or 3-phenylpropyl, 2-phenyl-2-methylethyl, 1-, 2-, 3- or 4-phenylbutyl, naphthylmethyl, 1- or 2-naphthylethyl. Particular preference is given to phenylmethyl, 1- or 2-phenylethyl.

A heterocycle having 2 to 12 carbon atoms and at least one atom from the group oxygen, sulfur or nitrogen in the ring generally consists of 1 to 3, preferably 1 or 2, rings. The heterocycle preferably contains 1 to 3, preferably 1 or 2 heteroatoms. Preference is given to furan, pyrrole, thiophene, indole, isoindole, benzofuran, isobenzofuran, benzothiophene, isobenzothiophene, pyrazole, imidazole, 1,3- or 1,2-oxazole, 1,3- or 1,2-thiazole, 1,3- or 1,2-benzimidazole, 1,3- or 1,2-benzoxazole, 1,3- or 1,2-benzothiazole, pyridine, pyrimidine, pyrazine, 1,2-, 1,3- or 1,4-oxazine, 1,2-, 1,3- or 1,4-thiazine, quinoline, isoquinoline, benzo-1,2-, -1,3- or -1,4-diazine or partially or completely saturated derivatives thereof, e.g. tetrahydrofuran, 1,3-dioxolane, pyrrolidine, pyrroline, 1,3- or 1,4-dioxane, piperidine, tetrahydro-2H-pyran, piperazine, oxirane or aziridine. Particular preference is given to furan, pyrrole, indole, imidazole, 1,3-thiazole, 1,3-benzothiazole, pyridine, pyrimidine, quinoline, isoquinoline or partially or completely saturated derivatives thereof, e.g. tetrahydrofuran, 1,3-dioxolane, pyrrolidine, 1,3- or 1,4-dioxane, piperidine or tetrahydro-2H-pyran.

A heteroalkyl radical having 2 to 12 carbon atoms is generally an alkyl radical substituted by a heterocyclyl radical. The alkyl radical preferably consists of 1 to 4 carbon atoms, particularly preferably of 1 or 2 carbon atoms. In particular, mention may be made of 2-, 3- or 4-pyridylmethyl or -ethyl, 2-, 3- or 4-tetrahydropyranylmethyl or -ethyl, 2- or 3-furanylmethyl or -ethyl, 2- or 3-thiophenylmethyl or -ethyl, 2- or 3-pyrrolylmethyl or -methyl, 2- or 4-imidazolylmethyl or -ethyl, 2-, 4- or 5-pyrimidylmethyl or -ethyl, 2- or 3-tetrahydrofuranylmethyl or -ethyl, 2-, 3- or 4-piperidinylmethyl or ethyl, 2- or 3-pyrrolidinylmethyl or -ethyl.

Substituents of said radicals can preferably represent hydrogen atoms, lower alkyl, hydroxyl, lower alkyloxy, thio, lower alkylthio, amino, lower alkylamino, di(lower alkyl)amino, nitro, iodine, bromine, fluorine, chlorine, azido, thiocyanato, isothiocyanato, cyanato, isocyanato, nitrile, isonitrile, phosphate, lower alkylphosphate, di(lower alkyl)phosphate, sulfonic acid, lower alkylsulfonate, sulfonamide, di(lower alkyl)sulfonamide or lower alkyl sulfonamide radicals. Particular preference is given to hydrogen atoms, lower alkyl, hydroxyl, lower alkyloxy, amino, di(lower alkyl)amino, chlorine, nitrile, sulfonic acid, sulfonamide or lower alkylsulfonate radicals.

The radicals can contain 1 to 10, preferably 1 to 5, particularly preferably 1 to 2, substituents.

Preference is given to cosmetic and/or dermatological preparations which comprise catechol oximes of the formula

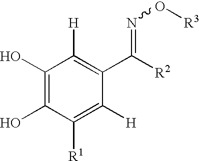

where

R¹ is hydrogen, methyl, tert-butyl, hydroxyl or methoxy, and

R² is hydrogen, an alkyl radical having 1 to 10 carbon atoms or an alkenyl radical having 2 to 10 carbon atoms, and

R³ is hydrogen, an optionally substituted alkyl radical having 1 to 10 carbon atoms, an optionally substituted alkenyl radical having 2 to 10 carbon atoms or an optionally substituted aryl or arylalkyl radical having 6 to 12 carbon atoms,

including stereoisomers thereof or mixtures thereof.

Particular preference is given to cosmetic and/or dermatological preparations which comprise catechol oximes of the formula

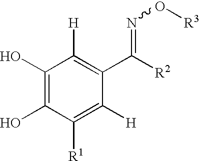

where

R¹ is hydrogen, hydroxyl or methoxy, and

R² is hydrogen, methyl, ethyl, ethenyl, isopropyl, propyl, tert-butyl, isobutyl or n-butyl, and

R³ is hydrogen, methyl, ethyl, ethenyl, isopropyl, propyl, tert-butyl, isobutyl, n-butyl, n-pentyl, isopentyl, prenyl, neopentyl, cyclopentyl, cyclohexyl, pentylmethyl, n-hexyl, n-heptyl, n-octyl, 2-ethylhexyl, n-nonyl, n-decyl, benzyl, 4-methylbenzyl, phenyl or 4-methylphenyl group,

including stereoisomers thereof or mixtures thereof.

Examples of individual compounds for the cosmetic and/or dermatological preparations according to the invention which may be mentioned are

3,4-dihydroxybenzaldehyde oxime

3,4-dihydroxyacetophenone oxime

3,4,5-trihydroxybenzaldehyde oxime

3,4-dihydroxybenzaldehyde O-ethyloxime

3,4-dihydroxybenzaldehyde O-(4-methylbenzyl)oxime

3,4-dihydroxyacetophenone O-ethyloxime

3,4,5-trihydroxybenzaldehyde O-ethyloxime.

The invention also provides for the use of the catechol oximes of the formula

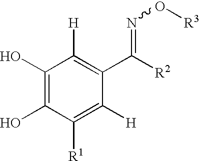

where

R¹ is hydrogen, lower alkyl or the group —O—R⁴ in which R⁴ is hydrogen or lower alkyl, and

R² is hydrogen, an alkyl radical having 1 to 22 carbon atoms or an alkenyl radical having 2 to 22 carbon atoms, and

R³ is hydrogen, an optionally substituted alkyl radical having 1 to 22 carbon atoms, an optionally substituted alkenyl radical having 2 to 22 carbon atoms, an optionally substituted aryl or arylalkyl radical having 6 to 12 carbon atoms or an optionally substituted heterocyclyl or heterocyclylalkyl radical having 2 to 12 carbon atoms and at least one atom from the group oxygen, sulfur or nitrogen,

including stereoisomers thereof or mixtures thereof, in cosmetic and/or dermatological preparations.

Some of said catechol oximes according to the invention are known.

The known catechol oximes according to the invention are described in Chem. Ber. 1922, 55, 920 to 929, in Chem. Ber. 1922, 55, 2357 to 2372 and in Liebigs Ann. 1936, 526, 277 to 294. Indications to an effect as antioxidants or free-radical scavengers and their use in cosmetic and/or dermatological preparations are not given.

Catechol oximes of the formula

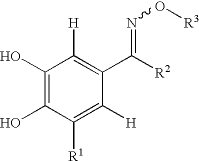

where

R¹ is hydrogen, lower alkyl or the group —O—R⁴ in which R⁴ is hydrogen or lower alkyl, and

R² is hydrogen, an alkyl radical having 1 to 22 carbon atoms or an alkenyl radical having 2 to 22 carbon atoms, and

R³ is an alkyl radical having 1 to 22 carbon atoms, an alkenyl radical having 2 to 22 carbon atoms, an optionally substituted aryl or arylalkyl radical having 6 to 12 carbon atoms,

including stereoisomers thereof or mixtures thereof are novel.

Preference is given to novel catechol oximes of the formula

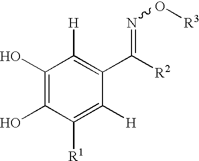

where

R¹ is hydrogen, methyl, tert-butyl, hydroxyl or methoxy, and

R² is hydrogen, an alkyl radical having 1 to 10 carbon atoms or an alkenyl radical having 2 to 10 carbon atoms, and

R³ is an alkyl radical having 1 to 10 carbon atoms, a substituted [lacuna] having 2 to 10 carbon atoms or an optionally substituted aryl or arylalkyl radical having 6 to 12 carbon atoms,

including stereoisomers thereof or mixtures thereof.

Particular preference is given to novel catechol oximes of the formula

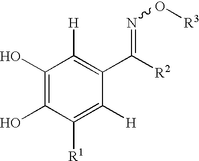

where

R¹ is hydrogen, hydroxyl or methoxy, and

R² is hydrogen, methyl, ethyl, ethenyl, isopropyl, propyl, tert-butyl, isobutyl or n-butyl, and

R³ is methyl, ethyl, ethenyl, isopropyl, propyl, tert-butyl, isobutyl, n-butyl, n-pentyl, isopentyl, prenyl, neopentyl, cyclopentyl, cyclohexyl, pentylmethyl, n-hexyl, n-heptyl, n-octyl, 2-ethylhexyl, n-nonyl, n-decyl, benzyl, 4-methylbenzyl, phenyl or 4-methylphenyl group,

including stereoisomers thereof or mixtures thereof.

Examples of individual compounds for the novel catechol oximes which may be mentioned are

3,4-dihydroxybenzaldehyde O-ethyloxime

3,4-dihydroxybenzaldehyde O-(4-methylbenzyl)oxime

3,4-dihydroxyacetophenone O-ethyloxime

3,4,5-trihydroxybenzaldehyde O-ethyloxime.

The preparations according to the invention can preferably be used in cosmetic or dermatological preparations for protecting cells and tissues of mammals, in particular humans, against the harmful effect of free radicals and reactive oxygen species. The preparations according to the invention can also be used analogously in other fields of use.

The amount of catechol oximes in the cosmetic or dermatological preparations according to the invention is 0.001% by weight to 30% by weight, preferably 0.001 to 20% by weight, particularly preferably 0.01% by weight to 5% by weight, based on the total weight of the preparation.

The preparation of the catechol oximes according to the invention is known per se (cf. Chem. Ber. 1922, 55, pages 920-929, in Chem. Ber. 1922, 55, pages 2357-2372 and Liebigs Ann. 1936, 526, pages 277-294) and can be carried out by reacting the corresponding aromatic carbonyl compound with hydroxylamines of the formula

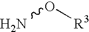

or ammonium salts thereof, where R³ has the meaning given above, in a solvent (e.g. water, an aliphatic mono- or polyhydric alcohol having 1 to 4 carbon atoms (such as e.g. methanol, ethanol, ethylene glycol, isopropanol, propanol, tert-butanol, n-butanol, isobutanol), 1,4-dioxane, tetrahydrofuran, N,N-dimethylformamide), preferably in water, methanol or ethanol, or in a mixture of solvents, optionally also together with one or more auxiliary bases (e.g. alkali metal hydroxides, alkali metal carbonates, alkali metal alkoxides, alkaline earth metal hydroxides, alkaline earth metal carbonates, alkaline earth metal alkoxides, alkaline earth metal oxides, basic inorganic or organic ion exchangers, ammonia, organic aliphatic amines, organic aromatic or heterocyclic amines), but preferably with sodium hydroxide, ammonia or sodium acetate, at −10° C. to 120° C., preferably at 20° C. to 100° C. The resulting catechol oximes according to the invention can then optionally be neutralized with a mineral acid and be purified using customary procedures (e.g. filtration, crystallization, chromatography, distillation), preferably by crystallization.

The process can be illustrated by the following equation:

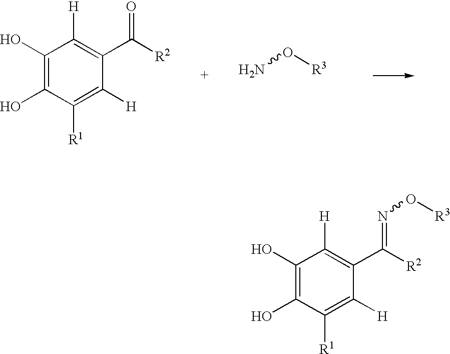

in which

R¹, R² and R³ have the meanings given above.

The aromatic carbonyl compounds used are preferably 3,4-dihydroxybenzaldehyde, 3,4,5-trihydroxybenzaldehyde or 3,4-dihydroxyacetophenone.

The hydroxylamines used are preferably hydroxylamine, O-ethylhydroxylamine or O-(4-methylbenzyl)hydroxylamine or the salts of said hydroxylamines.

The cosmetic and dermatological preparations according to the invention comprise the catechol oximes in an effective amount, in addition to other otherwise customary composition constituents. They comprise 0.001% by weight to 30% by weight, preferably 0.001 to 20% by weight, but in particular 0.01% by weight to 5% by weight, based on the total weight of the formulation, of the catechol oximes of the general formula I and can be in the form of “water in oil”, “oil in water”, “water in oil in water” or “oil in water in oil” emulsions, microemulsions, gels, solutions e.g. in oils, alcohols or silicone oils, sticks, soaps, aerosols, sprays or else foams. Further customary cosmetic auxiliaries and additives can be present in amounts of 5-99.99% by weight, preferably 10-80% by weight, based on the total weight of the formulation. In addition, the formulations can comprise water in an amount up to 99.99% by weight, preferably 5-80% by weight, based on the total weight of the formulation.

To prepare the cosmetic and dermatological preparations according to the invention, in a further embodiment, the catechol oximes according to the invention can also be incorporated beforehand into liposomes, e.g.starting from phosphatidylcholine, into microspheres, into nanospheres or else into capsules of a suitable matrix, e.g. of natural or synthetic waxes or of gelatin.

For use, the cosmetic and dermatological preparations according to the invention are applied to the skin and/or the hair in an adequate amount in the manner customary for cosmetics.

The cosmetic and dermatological preparations according to the invention can comprise cosmetic auxiliaries and additives, as are customarily used in such preparations, e.g. sunscreens (e.g. organic or inorganic light filter substances, preferably micropigments), preservatives, bactericides, fungicides, virucides, cooling active ingredients, plant extracts, antiinflammatory active ingredients, substances which accelerate wound healing (e.g. chitin or chitosan and derivatives thereof), film-forming substances (e.g. polyvinylpyrrolidones or chitosan or derivatives thereof), customary antioxidants, vitamins (e.g. vitamin C and derivatives, tocopherols and derivatives, vitamin A and derivatives), 2-hydroxycarboxylic acids (e.g. citric acid, malic acid, L-, D- or dl-lactic acid), skin lightening agents (e.g. kojic acid, hydroquinone or arbutin), skin coloring agents (e.g. walnut extracts or dihydroxyacetone), perfumes, antifoams, dyes, pigments which have a coloring action, thickeners, surface-active substances, emulsifiers, emollients, moisturizers and/or humectants (e.g. glycerol or urea), fats, oils, unsaturated fatty acids or derivatives thereof (e.g. linoleic acid, α-linolenic acid, γ-linolenic acid or arachidonic acid and their respective natural or synthetic esters), waxes or other customary constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives).

The amounts of cosmetic or dermatological auxiliaries and additives and perfume to be used in each case can be readily determined by simple experimentation by the person skilled in the art depending on the nature of the product in question.

The cosmetic and dermatological preparations according to the invention can preferably additionally comprise one or more of the catechol oximes according to the invention or else one or more other antioxidants. In particular, other antioxidants which may be used are all antioxidants customary or suitable for cosmetic and/or dermatological applications. The antioxidants are advantageously chosen from the group consisting of amino acids (e.g. glycine, histidine, 3,4-dihydroxyphenylalanine, tyrosine, tryptophan) and derivatives thereof, imidazoles (e.g. urocanic acid) and derivatives thereof, peptides (D,L-camosine, D-carnosine, L-carnosine, anserine) and derivatives thereof, carotenoids, carotenes (e.g. α-carotene, β-carotene, lycopene) and derivatives thereof, chlorogenic acid and derivatives thereof, lipoic acid and derivatives thereof, aurothioglucose, propylthiouracil and other thiols (e.g. thioredoxin, glutathione, cysteine, cystine, cystamine and the glycosyl and N-acyl derivatives thereof or alkyl esters thereof) and salts thereof, dilauryl thiodipropionate, distearyl thiodipropionate, thiodipropionic acid and derivatives thereof, and phenol acid amides of phenolic benzylamines (e.g. homovanillin acid amides, 3,4-dihydroxyphenylacetic acid amides, ferulic acid amides, sinapinic acid amides, caffeic acid amides, dihydroferulic acid amides, dihydrocaffeic acid amides, vanillomandelic acid amides or 3,4-dihydroxymandelic acid amides of 3,4-dihydroxybenzylamine, 2,3,4-trihydroxybenzylamine or 3,4,5-trihydroxybenzylamine), and also (metal) chelating agents (e.g. 2-hydroxy fatty acids, phytic acid, lactoferrin), humic acid, bile acids, bile extracts, bilirubin, biliverdin, folic acid and derivatives thereof, ubiquinone and ubiquinol and derivatives thereof, vitamin C and derivatives thereof (e.g. ascorbyl palmitate, magnesium ascorbyl phosphate, ascorbyl acetate), tocopherols and derivatives (e.g. vitamin-E acetate), vitamin A and derivatives (e.g. vitamin A palmitate), rutinic acid and derivatives thereof, flavonoids (e.g. quercetin, α-glucosylrutin) and derivatives thereof, phenol acids (e.g. gallic acid, ferulic acid) and derivatives thereof (e.g. propyl gallate, ethyl gallate, octyl gallate), furfurylideneglucitol, dibutylhydroxytoluene, butylhydroxyanisole, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, resveratrol) and the derivatives of said active ingredients which are suitable according to the invention.

The amount of further antioxidants in the preparations according to the invention can generally be 0.001 to 30% by weight, preferably 0.001 to 20% by weight, particularly preferably 0.001 to 5% by weight, based on the total weight of the preparation.

Apart from the catechol oximes according to the invention, two or more further antioxidants can of course be used.

In the cosmetic or dermatological preparations according to the invention, however, it is also possible to use UV-A and/or UV-B filter substances, where the total amount of filter substances can be 0.1 to 30% by weight, preferably 0.5 to 10% by weight, based on the total weight of the preparations, giving, for example, sunscreen compositions for skin and hair. UV-A and/or UV-B filter substances which can be used are, for example, 3-benzylidenecamphor derivatives (e.g. 3-(4-methyl-benzylidene)-dl-camphor), aminobenzoic acid derivatives (e.g. 2-ethylhexyl 4-(N,N-dimethylamino)benzoate or menthyl anthranilate), 4-methoxycinnamates (e.g. 2-ethylhexyl p-methoxycinnamate or isoamyl p-methoxycinnamate), benzophenones (e.g. 2-hydroxy-4-methoxybenzophenone), mono- or polysulfonated UV filters (e.g. 2-phenylbenzimidazole-5-sulfonic acid, sulisobenzo or 1,4-bis(benzimidazolyl)-benzene-4,4′,6,6′-tetrasulfonic acid or 3,3′-(1,4-phenylenedimethylidene)-bis-(7,7-dimethyl-2-oxo-bicyclo-[2.2.1]heptan-1-methanesulfonic acid) and salts thereof], salicylates (e.g. 2-ethylhexyl salicylate or homomenthyl salicylate), triazines {e.g. 2,4-bis-[4-(2-ethylhexyloxy)-2-hydroxyphenyl]-6-(4-methoxyphenyl)-1,3,5-triazine, bis(2-ethylhexyl) 4,4′-([6-([(1,1-dimethylethyl)-aminocarbonyl]phenylamino)-1,3,5-triazin-2,4-diyl]diimino)bisbenzoate}, 2-cyanopropenoic acid derivatives (e.g. 2-ethylhexyl 2-cyano-3,3-diphenyl-2-propenoate), dibenzoyl derivatives (e.g. 4-tert-butyl-4′-methoxydibenzoylmethane), polymer-bonded UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

The lipid phase in the cosmetic and/or dermatological preparations according to the invention can advantageously be chosen from the following groups of substances: mineral oils (advantageously paraffin oil), mineral waxes, hydrocarbons (advantageously squalane or squalene), synthetic or semisynthetic triglyceride oils (e.g. triglycerides of capric or caprylic acid), natural oils (e.g. castor oil, olive oil, sunflower oil, soybean oil, groundnut oil, rapeseed oil, almond oil, palm oil, coconut oil, palm kernel oil, borage seed oil and the like), natural ester oils (e.g. jojoba oil), synthetic ester oils (preferably esters of saturated and/or unsaturated, linear and/or branched alkanecarboxylic acids having 3 to 30 carbon atoms with saturated and/or unsaturated, linear and/or branched alcohols having 3 to 30 carbon atoms and esters of aromatic carboxylic acids with a saturated and/or unsaturated, linear and/or branched alcohols having 3 to 30 carbon atoms, in particular chosen from the group consisting of isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate, and synthetic or natural mixtures of such esters), fats, waxes and other natural and synthetic fatty substances, preferably esters of fatty alcohols with alcohols of low carbon number (e.g. with isopropanol, propylene glycol or glycerol) or esters of fatty alcohols with alkanoic acids of low carbon number or with fatty acids, alkyl benzoates (e.g. mixtures of n-dodecyl, n-tridecyl, n-tetradecyl and n-pentadecyl benzoate), and cyclic or linear silicone oils (such as, for example, dimethylpolysiloxanes, diethylpolysiloxanes, diphenylpolysiloxanes and mixed forms thereof).

The aqueous phase of the cosmetic and/or dermatological preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl ether, monoethyl or monobutyl ether, diethylene glycol monomethyl or monoethyl ether and analogous products, and also alcohols of low carbon number, e.g. ethanol, isopropanol, 1,2-propanediol, glycerol, and also α- or β-hydroxy acids, preferably lactic acid, citric acid or salicylic acid, and also emulsifiers which can advantageously be chosen from the group of ionic, nonionic, polymeric, phosphate-containing and zwitterionic emulsifiers, and in particular one or more thickeners which can advantageously be chosen from the group consisting of silicon dioxide, aluminum silicates, such as, for example, bentonites, polysaccharides and derivatives thereof, e.g. hyaluronic acid, guar seed flour, xanthan gum, hydroxypropylmethylcellulose or allulose derivatives, particularly advantageously from the group of polyacrylates, preferably a polyacrylate from the group of Carbopols, in each case individually or in combination or from the group of polyurethanes.

Particular preference is given to the use of the cosmetic or dermatological preparations according to the invention for protecting tissues and cells of mammals, in particular of the skin, the hair and/or the nails of humans, against oxidative stress and the harmful effect of free radicals.

The present invention likewise also covers a process for protecting cosmetic or dermatological preparations against oxidation or photooxidation, these preparations being, for example, preparations for the treatment, protection and care of the skin, the nails or the hair or, in addition, also make-up products whose constituents bring with them stability problems due to oxidation or photooxidation during storage, which involves the cosmetic or dermatological preparations having an effective content of catechol oximes according to the invention.

### EXAMPLES

Preparing the Preparation

### Example 1

### Cosmetic Solution [lacuna] 3,4-dihydroxybenzaldoxime

| | Content in |
|---|---|
| Raw material name | % by weight |
| 1,3-Butylene glycol | 99.9 |
| 3,4-Dihydroxybenzaldehyde oxime | 0.1 |

### EXAMPLE 2

### “Oil in Water” Emulsion Containing 3,4-dihydroxybenzaldoxime

| | | | Content |
|---|---|---|---|
| | | | in % by |
| Part | Raw material name (manufacturer) | Chemical name | weight |
| A | Arlatone 983 S ® (ICI) | Ether of poly- | 1.2 |
| | | ethylene glycol | |
| | | with glycerol | |
| | | monostearate | |
| | Brij 76 ® (ICI) | 3,6,9,12,15,18,21, | 1.2 |
| | | 24,27,30,33,36- | |
| | | Decaoxaoctatetra- | |
| | | contan-1-ol | |
| | Cutina MD ® (Henkel) | Glyceryl- | 3.5 |
| | | monostearate | |
| | Baysiloneöl M10 ® (GE Bayer) | Polydimethyl- | 0.8 |
| | | siloxane | |
| | Eutanol G ® (Henkel) | Octyldodecanol | 3.0 |
| | Paraffin oil 65 cp (Henry Lamotte) | Mineral oil | 8.0 |
| B | Water, dist. | | 49.8 |
| | Phenopip ® (Nipa Laboratories) | 2-Phenoxyethanol | 0.5 |
| | | and methyl | |
| | | 4-hydroxybenzoate | |
| | | and ethyl | |
| | | 4-hydroxybenzoate | |
| | | and propyl | |
| | | 4-hydroxybenzoate | |
| | | and butyl | |
| | | 4-hydroxybenzoate | |
| | 1,2-Propylene glycol | | 2.0 |
| | Glycerol 99% | | 3.0 |
| | 3,4-Dihydroxybenzaldehyde oxime | | 0.1 |
| C | Water, dist. | | 25.0 |
| | Carbopol 2050 ® (B. F. Goodrich) | Crosslinked acrylic | 0.4 |
| | | acid/C₁₀-C₃₀-alkyl | |
| | | acrylate polymer | |
| | Aqueous sodium hydroxide | | 1.2 |
| | solution, 10% | | |
| D | Perfume oil | | 0.3 |

Part A was mixed and heated to 80° C. Part B was mixed and heated to 90° C. and added to part A with stirring. For part C, Carbopol was carefully dispersed in water and neutralized with sodium hydroxide solution (pH 6.5). Part C was then added, at 60° C., to the mixture of parts A and B. Part D was added to the mixture of parts A, B and C at room temperature.

### EXAMPLE 3

### “Oil in Water” Emulsion Containing 3,4-dihydroxyacetophenone oxime

| | | | Content |
|---|---|---|---|
| | | | in % by |
| Part | Raw material name (manufacturer) | Chemical name | weight |
| A | Arlatone 983 S ® (ICI) | Ether of poly- | 1.2 |
| | | ethylene glycol | |
| | | with glycerol | |
| | | monostearate | |
| | Brij 76 ® (ICI) | 3,6,9,12,15,18,21, | 1.2 |
| | | 24,27,30,33,36- | |
| | | Decaoxaoctatetra- | |
| | | contan-1-ol | |
| | Cutina MD ® (Henkel) | Glyceryl- | 3.5 |
| | | monostearate | |
| | Baysiloneol M10 ® (GE Bayer) | Polydimethyl- | 0.8 |
| | | siloxane | |
| | Eutanol G ® (Henkel) | Octyldodecanol | 3.0 |
| | Paraffin oil 65 cp (Henry Lamotte) | Mineral oil | 8.0 |
| B | Water, dist. | | 49.8 |
| | Phenopip ® (Nipa Laboratories) | 2-Phenoxyethanol | 0.5 |
| | | and methyl | |
| | | 4-hydroxybenzoate | |
| | | and ethyl | |
| | | 4-hydroxybenzoate | |
| | | and propyl | |
| | | 4-hydroxybenzoate | |
| | | and butyl | |
| | | 4-hydroxybenzoate | |
| | 1,2-Propylene glycol | | 2.0 |
| | Glycerol 99% | | 3.0 |
| | 3,4-dihydroxybenzaldehyde oxime | | 0.2 |
| C | Water, dist. | | 25.0 |
| | Carbopol 2050 ® (B. F. Goodrich) | Crosslinked acrylic | 0.4 |
| | | acid/C₁₀-C₃₀-alkyl | |
| | | acrylate polymer | |
| | Aqueous sodium hydroxide | | 1.2 |
| | solution, 10% | | |
| D | Perfume oil | | 0.3 |

Part A was mixed and heated to 80° C. Part B was mixed and heated to 90° C. and added to part A with stirring. For part C, Carbopol was carefully dispersed in water and neutralized with sodium hydroxide solution (pH 6.5). Part C was then added, at 60° C., to the mixture of parts A and B. Part D was added to the mixture of parts A, B and C at room temperature.

### EXAMPLE 4

### “Water in Oil” Sunscreen Emulsion with UVA/B Broadband Protection and 3,4-dihydroxybenzaldoxime

| | | | Content |
|---|---|---|---|
| | Raw material | | in % by |
| Part | name (manufacturer) | Chemical name | weight |
| A | Dehymuls PGPH ® | Polyglycerol-2 | 3.0 |
| | (Henkel) | dipolyhydroxystearate | |
| | Monomuls 90-O 18 ® | Glyceryl oleate | 1.0 |
| | (Henkel) | | |
| | Permulgin 2550 ® (Koster | Beeswax | 1.0 |
| | Keunen Holland) | | |
| | Myritol 318 ® (Henkel) | Caprylic/caproic | 6.0 |
| | | triglycerides | |
| | Witconol TN ® (Witco) | C₁₂-C₁₅-Alkyl benzoate | 6.0 |
| | Cetiol SN ® (Henkel) | Cetyl and stearyl | 5.0 |
| | | isononanoate | |
| | Copherol 1250 ® (Henkel) | Tocopherol acetate | 1.0 |
| | Solbrol P ® (Bayer) | Propyl 4-hydroxybenzoate | 0.1 |
| | Neo Heliopan ® AV | 2-Ethylhexyl p- | 4.0 |
| | (Haarmann & Reimer) | methoxycinnamate | |
| | Neo Heliopan ® E | Isoamyl p- | 4.0 |
| | 1000 (Haarmann & Reimer) | methoxycinnamate | |
| | Neo Heliopan ® MBC | 3-(4-Methylbenzylidene)- | 2.0 |
| | (Haarmann & Reimer) | dl-camphor | |
| | Neo Heliopan ® OS | 2-Ethylhexyl salicylate | 3.0 |
| | (Haarmann & Reimer) | | |
| | Octyl triazone | | 1.0 |
| | Zinc oxide neutral | | 7.0 |
| | (Haarmann & Reimer) | | |
| B | Water, dist. | | 40 |
| | Phenoxyethanol | | 0.7 |
| | Solbrol M ® (Bayer) | Methyl 4-hydroxybenzoate | 0.2 |
| | Glycerol 99% | | 4.0 |
| | Neo Heliopan ® Hydro | 2-phenylbenzimidazole-5- | 10.0 |
| | (Haarmann & Reimer), | sulfonic acid | |
| | 15% as sodium salt | | |
| | Benzophenone-4 | | 0.5 |
| | 3,4-dihydroxybenzaldehyde | | 0.1 |
| | oxime | | |
| C | Perfume oil | | 0.3 |
| | Bisabol | | 0.1 |

For part A, all substances apart from the zinc oxide were heated to 85° C. and the zinc oxide was carefully dispersed in the mixture. The components of part B were mixed, heated to 85° C. and added to part A with stirring. Part C was added to the mixture of parts A and B and then the mixture was homogenized using a dispersion tool.

### EXAMPLE 5

### “Oil in Water” Sunscreen Emulsion with UVA/B Broadband Protection and 3,4-dihydroxybenzaldehyde oxime

| | | | Content |
|---|---|---|---|
| | Raw material | | in % by |
| Part | name (manufacturer) | Chemical name | weight |
| A | Arlacel 165 ® (ICI) | Glyceryl stearate and | 3.0 |
| | | polyethylene glycol 100 | |
| | | stearate | |
| | Emulgin B2 ® (Henkel) | Ceteareth-20 | 1.0 |
| | Lanette O ® (Henkel) | Cetyl and stearyl alcohol | 1.15 |
| | Myritol 318 ® (Henkel) | Caprylic/caproic | 5.0 |
| | | triglycerides | |
| | Cetiol SN ® (Henkel) | Cetyl and stearyl | 4.0 |
| | | isononanoate | |
| | Abil 100 ® (Goldschmidt) | Polydimethylsiloxane | 1.0 |
| | Bentone Gel MIO ® | Mineral oil and | 3.0 |
| | (Rheox) | quaternium-18 | |
| | | hectorite and propylene | |
| | | carbonate | |
| | Cutina CBS ® (Henkel) | Glyceryl stearate and cetyl | 2.0 |
| | | alcohol and stearyl alcohol | |
| | | and cetyl palmitate and | |
| | | cocoglycerides | |
| | Neo Heliopan ® 303 | 2-Ethylhexyl 2-cyano-3,3- | 7.0 |
| | (Haarmann & Reimer) | diphenyl-2-propenoate | |
| | Neo Heliopan ® BB | 2-Hydroxy-4-methoxy- | 1.0 |
| | (Haarmann & Reimer) | benzophenone | |
| | Neo Heliopan ® MA | Menthyl anthranilate | 3.0 |
| | (Haarmann & Reimer) | | |
| | 2-ethylhexyl N,N-dimethyl- | | 3.0 |
| | 4-aminobenzoate | | |
| | Titanium dioxide, microfine | | 5.0 |
| B | Water, dist. | | 55.85 |
| | Veegum ultra ® | Magnesium aluminum | 1.0 |
| | (Vanderbilt) | sulfate | |
| | Natrosol 250 HHR | Hydroxymethylcellulose | 0.3 |
| | (Hercules) | | |
| | Glycerol | | 3.0 |
| | Phenopip ® | 2-Phenoxyethanol | 0.3 |
| | (Nipa Laboratories) | and methyl | |
| | | 4-hydroxybenzoate | |
| | | and ethyl | |
| | | 4-hydroxybenzoate | |
| | | and propyl | |
| | | 4-hydroxybenzoate | |
| | | and butyl | |
| | | 4-hydroxybenzoate | |
| | 3,4-Dihydroxybenzalde- | | 0.1 |
| | hyde oxime | | |
| C | Perfume oil | | 0.3 |

For part A, all substances apart from the titanium dioxide were mixed and heated to 85° C.; the titanium dioxide was carefully dispersed into the mixture. For part B, all substances apart from Veegum and Natrosol were mixed, heated to 90° C., Natrosol and Veegum were dispersed therein and the mixture was added, with stirring, to part A. Part C was added to the mixture of parts A and B and then the mixture was homogenized using a dispersion tool (pH 5.6).

### EXAMPLE 6

### “Oil in Water” Sunscreen Emulsion with UVA/B Broadband Protection and 3,4-dihydroxybenzaldehyde oxime

| | | | Content |
|---|---|---|---|
| | Raw material | | in % by |
| Part | name (manufacturer) | Chemical name | weight |
| A | Crodaphos MCA ® | Cetyl phosphate | 1.50 |
| | (Croda) | | |
| | Cutina MD ® (Henkel) | Glyceryl stearate | 2.0 |
| | Lanette 16 ® (Henkel) | Cetyl alcohol | 1.2 |
| | Myritol 318 ® (Henkel) | Caprylic/caproic | 5.0 |
| | | triglycerides | |
| | Cetiol SN ® (Henkel) | Cetyl and stearyl | 5.0 |
| | | isononanoate | |
| | Copherol 1250 ® (Henkel) | Tocopherol acetate | 0.5 |
| | Solbrol ® P (Bayer) | Propyl 4-hydroxybenzoate | 0.1 |
| | Abil 100 ® (Goldschmidt) | Polydimethylsiloxane | 0.3 |
| | Neo Heliopan ® HMS | 3,3,5-Trimethylcyclohexyl | 5.0 |
| | (Haarmann & Reimer) | salicylate | |
| | Butylmethoxydibenzoyl- | | 2.0 |
| | methane | | |
| B | Water, dist. | | 47.8 |
| | 1,3-Butylene glycol | | 3.0 |
| | Sobrol ® M (Bayer) | Methyl 4-hydroxybenzoate | 0.2 |
| | Phenoxyethanol | | 0.7 |
| | Carbopol ETD 2050 ® | Copolymer acrylic acid/ | 0.2 |
| | (B. F. Goodrich) | C₁₀-C₃₀-alkylacrylate | |
| | Keltrol T ® (Calgon) | Xanthan gum | 0.2 |
| | Neo Heliopan ® AP | 2,2-(1,4-Phenylene)-bis- | 22 |
| | (Haarmann & Reimer) | (1H-benzimidazole-4,6- | |
| | | disulfonic acid and | |
| | | disodium salt | |
| | 3,4-Dihydroxybenzalde- | | 0.1 |
| | hyde oxime | | |
| C | Aqueous sodium hydroxide | | 2.8 |
| | solution, 10% | | |
| D | Perfume oil | | 0.3 |
| | Bisabolol | | 0.1 |

Part A was heated to 85° C. Part B: Carbopol and Keltrol were dispersed into the residual constituents in the cold, the mixture was heated to 85° C. and added to part A. Part C was immediately added, at 80° C., to the mixture of parts A and B and homogenized for 5 min using a dispersion tool. Part D was then added at room temperature and the mixture was homogenized using a dispersion tool (pH 6.6).

Preparation Process for Catechol Oximes

The carbonyl compound (87 mmol) was dissolved in 45 ml of water at 40° C. A solution of the corresponding hydroxylamine hydrochloride (90 mmol) and of sodium acetate (87 mmol) in 25 ml of water were added, and the reaction mixture was stirred at about 80° C. for 2 h under nitrogen. After cooling, the mixture was extracted with 200 ml of tert-butyl methyl ether, the organic phase was washed with saturated sodium chloride solution, dried over sodium sulfate and filtered, and the filtrate was evaporated to dryness under reduced pressure. The crystalline residue was optionally recrystallized.

TABLE 1

| Examples | Name | CAS No. | m.p./° C. | MS |
|---|---|---|---|---|
| 7 | 3,4-Dihydroxy- | 3343-59-7 | 143 | ESI-(−): m/e = |
| | benzaldehyde | | (decomp.) | 305.0 (100%), |
| | oxime | | | 152.2 (80%) |
| 8 | 3,4-Dihydroxy- | Beilstein | 135 | APCI-(−): |
| | acetophenone | ref. No. | (decomp.) | m/e = 166.4 |
| | oxime | 3249378 | | (100%), 332.7 |
| | | | | (11%) |
| 9 | 3,4,5-Trihydroxy- | 53148-14-4 | 177 | APCI-(+): |
| | benzaldehyde | | (decomp.) | m/e = 170.0 |
| | oxime | | | (100%), 154.3 |
| | | | | (11%) |
| 10 | 3,4-Dihydroxy- | — | <23 | EI: m/e = 181 |
| | benzaldehyde | | | (100%), 153 |
| | O-ethyloxime | | | (20%), 152 |
| | | | | (19%), 136 |
| | | | | (28%), 126 |
| | | | | (26%), 110 |
| | | | | (47%), 109 |
| | | | | (30%), 81 |
| | | | | (18%), 53 |
| | | | | (14%), 29 |
| | | | | (16%) |
| 11 | 3,4-Dihydroxy- | — | 85-86 | APCI-(−): |
| | benzaldehyde | | | m/e = 256.3 |
| | O-(4-methyl- | | | (100%) |
| | benzyl)oxime) | | | |
| 12 | 3,4-Dihydroxy- | — | 112 | ESI-(−): m/e = |
| | acetophenone | | | 194.0 (100%), |
| | O-ethyloxime | | | 388.7 (77%) |
| 13 | 3,4,5-Trihydroxy- | — | 80 | EI: m/e = 197 |
| | benzaldehyde | | | (100%), 153 |
| | O-ethyloxime | | | (17%), 152 |
| | | | | (29%), 142 |
| | | | | (30%), 126 |
| | | | | (47%), 125 |
| | | | | (33%), 96 |
| | | | | (17%), 79 |
| | | | | (28%), 51 |
| | | | | (17%) |

Demonstration of Activity

### EXAMPLE 14

### Activity as Free-radical Scavenger

The activity of the illustrative compounds as in examples 7 to 13 as free-radical scavengers was compared with the conventional free-radical scavenger and two examples from WO 95 01,157. For this, the DPPH (1,1-diphenyl-2-picryl-hydrazyl) test was used to eliminate free radicals.

DPPH was dissolved in methanol to a concentration of 100 μmol/l. A series of dilutions of the illustrative compounds, vitamin C, α-tocopherol and dibutylhydroxytoluene were prepared in methanol. Methanol was used as the control. 2500 μl of the DPPH solution were mixed with 500 μl of each test solution, and the decrease in the absorption at 515 nm was read off until the decrease was less than 2% per hour. The activity of the test substances as free-radical scavengers was calculated according to the following equation:

Activity as free-radical scavenger (%)=100−(absorption of the test compounds)/(absorption of the control)×100.

The activity of the free-radical scavengers (%) in a series of dilutions of test compounds was used to calculate, for each test compound, the effective relative concentration EC₅₀ (based on the initially present concentration of DPPH, EC=c (test compound)/c(DPPH)) of a test compound at which 50% of the DPPH free radical had been eliminated. The results are given in Table 2:

TABLE 2

| Test compound | |
|---|---|
| according to example | EC₅₀/(mol/mol) |
| 7 | 0.053 |
| 8 | 0.073 |
| 9 | 0.090 |
| 10 | 0.156 |
| 11 | 0.131 |
| 12 | 0.298 |
| 13 | 0.132 |
| Vitamin C | 0.270 |
| α-Tocopherol | 0.250 |
| Dibitylhydroxytoluene | 0.240 |
| Comparison: | |
| examples from WO 95 01,157 | |
| Salicylaldoxime | >3.0 |
| o-Hydroxyacetophenone oxime | >3.0 |

### EXAMPLE 15

### Activity as Antioxidants

The activity of the illustrative compounds according to Examples 7 to 13 as antioxidants was compared with the conventional antioxidants and two examples from WO 95 01,157. The test system used was the accelerated autoxidation of lipids by air with or without antioxidant using the Rancimat apparatus (Rancimat is a registered trademark of Metrohm AG, Herisau, Switzerland).

The illustrative compounds, vitamin C, α-tocopherol and dibutyihydroxytoluene were dissolved in methanol or acetone and 100 μl of each test solution were added to a prepared oil sample of 3 g. In a control sample only solvent was added. A constant stream of dry air (20 l/h) was bubbled through the heated oil sample containing the test solution, and the volatile oxidation products (predominantly short-chain fatty acids such as formic acid or acetic acid) were collected in a receiver containing water. The conductivity of this aqueous solution was measured continuously and documented. The oxidation of (unsaturated) fats proceeds here only very slowly for a period of time and then suddenly increases greatly. The time to the increase is referred to as the induction period (IP).

The antioxidative index (AOI) was obtained according to the following equation:

AOI=IP_{(with test solution)}/IP_{(control sample)}.

The results for the experiment at 100° C. in soybean oil that has been purified over alumina grade N are shown in table 3:

TABLE 3

| Test compound | AOI in soybean oil at 100° C. |
|---|---|
| according to example | with 0.05% of test substance |
| 7 | 15.8 |
| 8 | 16.6 |
| 9 | 20.9 |
| 10 | 17.3 |
| 11 | 14.1 |
| 12 | 15.2 |
| 13 | 17.2 |
| Vitamin C | 1.2 |
| α-Tocopherol | 5.1 |
| Dibutylhydroxytoluene | 4.8 |
| Comparison: | |
| examples from WO 95 01,157 | |
| Salicylaldoxime | 1.1 |
| o-Hydroxyacetophenone oxime | 0.9 |

The results for the experiment at 80° C. in squalene that was purified over alumina grade N and stabilized with 1 ppm of α-tocopherol are shown in table 4:

TABLE 4

| Test compound | AOI in squalene at 80° C. |
|---|---|
| according to example | with 0.005% of test substance |
| 7 | 70 |
| 8 | 95 |
| 9 | 150 |
| 10 | 85 |
| 11 | 50 |
| 12 | 65 |
| 13 | 126 |
| Vitamin C | 0.7 |
| α-Tocopherol | 39 |
| Dibutylhydroxytoluene | 38 |
| Comparison: | |
| examples from WO 95 01,157 | |
| Salicylaldoxime | 1.6 |
| o-Hydroxyacetophenone oxime | 1.6 |

### EXAMPLE 16

### Determination of the Protective Action Against Ultraviolet Light-induced Sebum Oxidation

One dose of 2 mg/cm² of the preparation from example 1 was applied twice daily to the skin on the back of 12 test persons for 2 days. Prior to the subsequent irradiation, a 0.2% strength ethanolic solution of [lacuna] was applied to a control area (2 mg/cm²). The 2 treated and one untreated site were irradiated with ultraviolet light (320 to 400 nm, 10 joule/cm²). The test areas in each case were treated for 2 min with 4 ml of ethanol in each case, the solutions were dried at room temperature under nitrogen and the residue was taken up in 1 ml of ethanol. The latter solutions were tested by HPLC for their content of squalene (detection at 210 nm against standard) or squalene hydroperoxide (SQOOH, determination of the peroxide content by means of cytochrome C/luminol-enhanced cheiniluminescence). The content of squalene peroxide was given relative to squalene in the form of picomoes of peroxide per μg of squalene.

The inhibition based on the untreated area was calculated using the following equation:

% inhibition=100·(c_{SQOOH, untreated}−c_{SQOOH, treated})/C_{SQOOH, untreated}

TABLE 5

| | c(H₂O₂)/c(SQ) | % inhibition compared |
|---|---|---|
| Skin area, treated with: | [pmol/μg] | with the untreated area |
| untreated | 930 ± 65 | — |
| Preparation according to | 565 ± 25 | 39 ± 4 |
| example 1 | | |
| 0.2% tocopherol in ethanol | 664 ± 19 | 28 ± 6 |

1. A cosmetic and/or dermatological preparation comprising catechol oximes of the formula 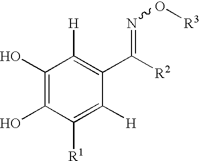
where
R¹ is hydrogen, lower alkyl or the group —O—R⁴ in which R⁴ is hydrogen or lower alkyl, and
R² is hydrogen, an alkyl radical having 1 to 22 carbon atoms or an alkenyl radical having 2 to 22 carbon atoms, and
R³ is hydrogen, an optionally substituted alkyl radical having 1 to 22 carbon atoms, an optionally substituted alkenyl radical having 2 to 22 carbon atoms, an optionally substituted aryl or arylalkyl radical having 6 to 12 carbon atoms or an optionally substituted heterocyclyl or heterocyclylalkyl radical having 2 to 12 carbon atoms and at least one atom from the group oxygen, sulfur or nitrogen,
including stereoisomers thereof or mixtures thereof.

2. The cosmetic and/or dermatological preparation comprising catechol oximes of the formula 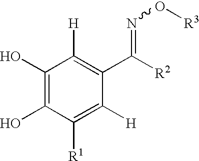
where
R¹ is hydrogen, methyl, tert-butyl, hydroxyl or methoxy, and
R² is hydrogen, an alkyl radical having 1 to 10 carbon atoms or an alkenyl radical having 2 to 10 carbon atoms, and
R³ is hydrogen, an optionally substituted alkyl radical having 1 to 10 carbon atoms, an optionally substituted alkenyl radical having 2 to 10 carbon atoms or an optionally substituted aryl or arylalkyl radical having 6 to 12 carbon atoms,
including stereoisomers thereof or mixtures thereof.

3. The cosmetic and/or dermatological preparation comprising catechol oximes of the formula 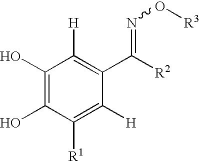
where
R¹ is hydrogen, hydroxyl or methoxy, and
R² is hydrogen, methyl, ethyl, ethenyl, isopropyl, propyl, tert-butyl, isobutyl or n-butyl, and
R³ is hydrogen, methyl, ethyl, ethenyl, isopropyl, propyl, tert-butyl, isobutyl, n-butyl, n-pentyl, isopentyl, prenyl, neopentyl, cyclopentyl, cyclohexyl, pentylmethyl, n-hexyl, n-heptyl, n-octyl, 2-ethylhexyl, n-nonyl, n-decyl, benzyl, 4-methylbenzyl, phenyl or 4-methylphenyl group,
including stereoisomers thereof or mixtures thereof.

4. The cosmetic and/or dermatological preparation comprising 3,4-dihydroxybenzaldehyde oxime, 3,4-dihydroxyacetophenone oxime, 3,4,5-trihydroxybenzaldehyde oxime, 3,4-dihydroxybenzaldehyde O-ethyloxime, 3,4-dihydroxybenzaldehyde O-(4-methylbenzyl)oxime, 3,4-dihydroxyacetophenone O-ethyloxime or 3,4,5-trihydroxybenzaldehyde O-ethyloxime.

5. The cosmetic and/or dermatological preparation comprising 0.001% by weight to 30% by weight, preferably 0.001 to 20% by weight, particularly preferably 0.01 to 5% by weight, of the catechol oximes as claimed in claims 1 to 4, based on the total weight of the preparations.

6. The use of the catechol oximes of the formula 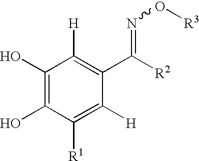
where
R¹ is hydrogen, lower alkyl or the group —O—R⁴ in which R⁴ is hydrogen or lower alkyl, and
R² is hydrogen, an alkyl radical having 1 to 22 carbon atoms or an alkenyl radical having 2 to 22 carbon atoms, and
R³ is hydrogen, an optionally substituted alkyl radical having 1 to 22 carbon atoms, an optionally substituted alkenyl radical having 2 to 22 carbon atoms, an optionally substituted aryl or arylalkyl radical having 6 to 12 carbon atoms or an optionally substituted heterocyclyl or heterocyclylalkyl radical having 2 to 12 carbon atoms and at least one atom from the group oxygen, sulfur or nitrogen,
including stereoisomers thereof or mixtures thereof, in cosmetic and/or dermatological preparations.

7. The use of the catechol oximes of the formula 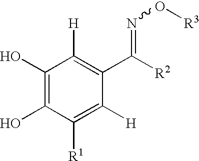
where
R¹ is hydrogen, methyl, tert-butyl, hydroxyl or methoxy, and
R² is hydrogen, an alkyl radical having 1 to 10 carbon atoms or an alkenyl radical having 2 to 10 carbon atoms, and
R³ is hydrogen, an optionally substituted alkyl radical having 1 to 10 carbon atoms, an optionally substituted alkenyl radical having 2 to 10 carbon atoms or an optionally substituted aryl or arylalkyl radical having 6 to 12 carbon atoms,
including stereoisomers thereof or mixtures thereof, in cosmetic and/or dermatological preparations.

8. The use of the catechol oximes of the formula 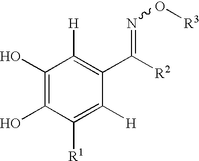
where
R¹ is hydrogen, hydroxyl or methoxy, and
R² is hydrogen, methyl, ethyl, ethenyl, isopropyl, propyl, tert-butyl, isobutyl or n-butyl, and
R³ is hydrogen, methyl, ethyl, ethenyl, isopropyl, propyl, tert-butyl, isobutyl, n-butyl, n-pentyl, isopentyl, prenyl, neopentyl, cyclopentyl, cyclohexyl, pentylmethyl, n-hexyl, n-heptyl, n-octyl, 2-ethylhexyl, n-nonyl, n-decyl, benzyl, 4-methylbenzyl, phenyl or 4-methylphenyl group,
including stereoisomers thereof or mixtures thereof, in cosmetic and/or dermatological preparations.

9. The use of 3,4-dihydroxybenzaldehyde oxime, 3,4-dihydroxyacetophenone oxime, 3,4,5-trihydroxybenzaldehyde oxime, 3,4-dihydroxybenzaldehyde O-ethyloxime, 3,4-dihydroxybenzaldehyde O-(4-methylbenzyl)oxime, 3,4-dihydroxyacetophenone O-ethyloxime or 3,4,5-trihydroxybenzaldehyde O-ethyloxime including stereoisomers thereof or mixtures thereof in cosmetic and/or dermatological preparations.

10. The use of the preparations as claimed in claims 1 to 5 as antioxidants and/or free-radical scavengers.

11. The use as claimed in claims 6 to 9 as antioxidants and/or free-radical scavengers.

12. The preparation as claimed in claims 1 to 5 which comprises, as additives, at least one UVA and/or UVB filter substance.

13. The preparation as claimed in claims 1 to 5, which comprises, as additives, at least one further antioxidant or a free-radical scavenger.

14. The preparation as claimed in claims 1 to 5, which comprises, as additives, at least one UVA and/or UVB filter substance and at least one further antioxidant or a free-radical scavenger.

15. A catechol oxime of the formula 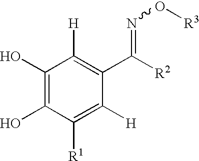
where
R¹ is hydrogen, lower alkyl or the group —O—R⁴ in which R⁴ is hydrogen or lower alkyl, and
R² is hydrogen, an alkyl radical having 1 to 22 carbon atoms or an alkenyl radical having 2 to 22 carbon atoms, and
R³ is an alkyl radical having 1 to 22 carbon atoms, an alkenyl radical having 2 to 22 carbon atoms, an optionally substituted aryl or arylalkyl radical having 6 to 12 carbon atoms,
including stereoisomers thereof or mixtures thereof.

16. A catechol oxime of the formula 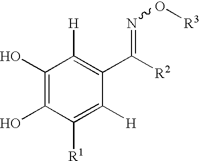
where
R¹ is hydrogen, methyl, tert-butyl, hydroxyl or methoxy, and
R² is hydrogen, an alkyl radical having 1 to 10 carbon atoms or an alkenyl radical having 2 to 10 carbon atoms, and
R³ is an alkyl radical having 1 to 10 carbon atoms, a substituted [lacuna] having 2 to 10 carbon atoms or an optionally substituted aryl or arylalkyl radical having 6 to 12 carbon atoms,
including stereoisomers thereof or mixtures thereof.

17. A catechol oxime of the formula 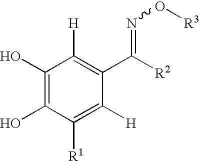
where
R¹ is hydrogen, hydroxyl or methoxy, and
R² is hydrogen, methyl, ethyl, vinyl, isopropyl, propyl, tert-butyl, isobutyl or n-butyl, and
R³ is methyl, ethyl, vinyl, isopropyl, propyl, tert-butyl, isobutyl, n-butyl, n-pentyl, isopentyl, prenyl, neopentyl, cyclopentyl, cyclohexyl, pentylmethyl, n-hexyl, n-heptyl, n-octyl, 2-ethylhexyl, n-nonyl, n-decyl, benzyl, 4-methylbenzyl, phenyl or 4-methylphenyl group,
including stereoisomers thereof or mixtures thereof.

18. A catechol oxime chosen from the group comprising 3,4-dihydroxybenzaldehyde O-ethyloxime, 3,4-dihydroxybenzaldehyde O-(4-methylbenzyl)oxime, 3,4-dihydroxyacetophenone O-ethyloxime and 3,4,5-trihydroxybenzaldehyde O-ethyloxime including stereoisomers thereof or mixtures thereof.

19. A process for the preparation of the catechol oximes of the formula 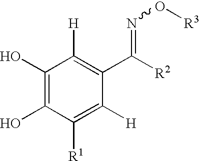
where
R¹ is hydrogen, lower alkyl or the group —O—R⁴ in which R⁴ is hydrogen or lower alkyl, and
R² is hydrogen, an alkyl radical having 1 to 22 carbon atoms or an alkenyl radical having 2 to 22 carbon atoms, and
R³ is an alkyl radical having 1 to 22 carbon atoms, an alkenyl radical having 2 to 22 carbon atoms, an optionally substituted aryl or arylalkyl radical having 6 to 12 carbon atoms,
characterized in that aromatic carbonyl compound of the formula 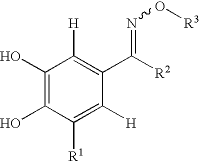
[lacuna] reacted with hydroxylamines of the formula,
where R¹ and R² have the meaning given above, 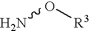
or ammonium salts thereof,
where R³ has the meaning given above,
in a solvent optionally also together with one or more auxiliary bases at −10° C. to 120° C., then optionally neutralized with a mineral acid and purified in the manner known per se.

20. The process as claimed in claim 19, characterized in that the carbonyl compounds used are 3,4-dihydroxybenzaldehyde, 3,4,5-trihydroxybenzaldehyde or 3,4-dihydroxyacetophenone.

21. The process as claimed in claims 19 and 20, characterized in that the O-alkylhydroxylamines used are O-ethylhydroxylamine or O-(4-methylbenzyl)-hydroxylamine or the salts of said hydroxylamines.
